(19) United States
(12) Patent Application Publication
(10) Pub. No.: US 2013/0064888 A1
(43) Pub. Date: Mar. 14, 2013

(54) PHARMACEUTICAL FORMULATIONS

(71) Applicants: Roey SOLOMONOVICH, Tel Aviv (IL); Dafna ARIELI, Kadima (IL)

(72) Inventors: Roey SOLOMONOVICH, Tel Aviv (IL); Dafna ARIELI, Kadima (IL)

(21) Appl. No.: 13/569,452

(22) Filed: Aug. 8, 2012

Related U.S. Application Data
(60) Provisional application No. 61/521,189, filed on Aug. 8, 2011.
(60) Provisional application No. 61/641,637, filed on May 2, 2012.

(51) Int. Cl. A61K 9/28; A61P 43/00; A61K 31/5377

(52) U.S. Cl. 424/465; 514/230.8

(57) ABSTRACT

The present invention provides a pharmaceutical dosage form in the form of a tablet comprising: (a) a compressed inert core, (b) an optional subcoat over the compressed inert core, (c) a drug layer over the compressed core (a) or optional subcoat (b) comprising a drug having a water solubility at 25° C. of about 100 mg/l or less, a coating polymer and optionally a surfactant, and (d) optionally one or more layers coating the drug layer. The present invention also provides a process of making the same.

### CROSS REFERENCE TO RELATED APPLICATIONS

This application claims the benefit of U.S. Provisional Application Nos. 61/521,189, filed Aug. 8, 2011, and 61/641,637, filed May 2, 2012, the contents of which are incorporated herein by reference.

### FIELD OF THE INVENTION

The present invention relates to pharmaceutical formulations and processes for their preparation. In particular the pharmaceutical formulations contain drugs that are practically insoluble in water. The formulations may provide immediate or modified release profiles of the drugs.

### BACKGROUND OF THE INVENTION

Drugs that have a low solubility in water, and particularly drugs that are practically insoluble in water (for example drugs having a water solubility of water solubility at 25° C. of about 100 mg/l or less), present challenges to the preparation of pharmaceutical formulations. In particular, achieving an acceptable dissolution rate and oral bioavailability can be difficult.

Rivaroxaban is a highly selective direct factor Xa inhibitor having anticoagulant activity, and can be used in the prophylaxis and treatment of thromboembolic diseases. Rivaroxaban has the formula:

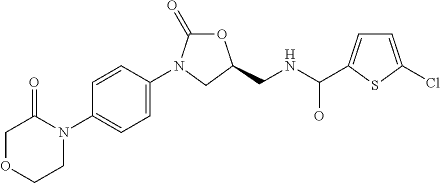 [SMILES: O=C1COCCN1c1ccc(N2C[C@H](CNC(O)c3ccc(Cl)s3)OC2=O)cc1]

and the chemical name: 5-chloro-N-({(5S)-2-oxo-3-[4-(3-oxo-4-morpholinyl)-phenyl]-1,3-oxaolidin-5-yl}methyl-2-thiophencarboxamide. Rivaroxaban is marketed as a film-coated tablet in a 10 mg dose under the name Xarelto® for use in the prevention of venous thromboembolism (VTE) in adult patients undergoing elective hip or knee replacement surgery. The listed excipients for Xarelto® tablets (excluding the film-coat) are microcrystalline cellulose, croscarmellose sodium, lactose monohydrate, hypromellose, sodium lauryl sulfate, and magnesium stearate. Rivaroxaban, in common with some other direct factor Xa inhibitors, is practically insoluble in water (<100 mg/l at 25° C.), and moreover has a low solubility in many organic solvents, including ethanol, and hence presents significant challenges to formulators. Further, since rivaroxaban is a low dose drug, there are further challenges as to achieving uniform distribution of the drug in a tablet.

The prior art discloses various approaches for formulating rivaroxaban. US 2008/0026057 discloses a process whereby the rivaroxaban is provided as granules prepared by wet granulation. In particular the granules are prepared by wet granulation of rivaroxaban with a hydrophilic binding agent and optionally a wetting agent in a solvent such as ethanol, acetone, water, or mixtures thereof.

The granules are dried and converted into a dosage form in subsequent steps, e.g. by sieving and compressing the granules to form tablets, or by filling the granules in capsules or sachets. However, the use of wet granulation is not particularly desirable in view of the need to remove solvent from the granules, which is an energy intensive process. Moreover, the granules prepared by the disclosed process have a slow disintegration. US 2010/0151011 discloses solid pharmaceutical dosage forms of rivaroxaban in multiparticulate form, which can be prepared by melting the active agent with one or more excipients. The process yields a melt or melt extrudate which, following milling, forms granules or powders that can be encapsulated, or further processed with other excipients to form granulates that can be compressed into tablets. However, melt processing is not a particularly desirable procedure as it restricts the excipients that can be used and further entails operation at suitably high temperatures to enable the production of a melt. This increases the risk of drug decomposition and polymorphic changes, as well as drug-excipient reactions, potentially leading to the presence of decomposition products in the final dosage form. US 2010/0151011 also discloses a method whereby rivaroxaban is dissolved together with an excipient (polyvinylpyrrolidone) in glacial acetic acid at high temperature, distilled, and dried. The resulting granules are ground and sieved. As discussed above, this method suffers from fact that there is a lack of suitable solvents that can be used to dissolve rivaroxaban. Acetic acid is a high boiling solvent that needs to be removed by evaporation. Hence, this process is highly energy intensive, and is not suitable for large scale manufacture.

WO 2010/003641 discloses pharmaceutical compositions of rivaroxaban comprising a solubilizer and a pseudo-emulsifier as excipients. The solubilizer can be a surfactant, and the pseudo-emulsifier is a natural product, such as a natural gum. The compositions can be prepared by dry granulation, by pellet layering to form a multiparticulate, by melting followed by grinding, or by co-precipitation with an antisolvent. These processes are said to form primary pharmaceutical compositions in the form of granules which are then further processed into a dosage form by mixing with further excipients and compressing to provide tablets. According to the disclosure of this publication, the compositions are preferably immediate release formulations. The processes disclosed in this publication involve the production of an intermediate product, namely granules before these are compressed to form a tablet, and hence involve multiple steps. Moreover, processes such a co-precipitation use large volumes of solvent, which is not economical, nor desirable, from an environmental perspective.

WO 2010/146179 discloses solid pharmaceutical compositions of rivaroxaban, prepared by dry mixing or dry granulation of the rivaroxaban with at least one excipient, co-milling rivaroxaban with the excipients, hot melt granulation with a molten excipient, or hot melt extrusion with an excipient. The mixture may then be agglomerated, granulated with a granulation liquid, or milled before compressing to form a tablet. As discussed above, melt processing is not a desirable process for large scale manufacture in view of the energy requirements and the potential for prolonged heating to cause degradation of the active agent. Further, co-milling is a very energy intensive process. Moreover optimum blend uniformity can be difficult to achieve using co-milling and dry granulation processes.

The methods described in the prior art and utilised in commercial products involve undesired steps that raise significant disadvantages to the overall tablet preparation process. It would therefore be desirable to provide compositions of drugs that have low water solubility, or drugs that are practically insoluble in water wherein the compositions have good blend uniformity, and which can achieve consistent release and dissolution profiles and moreover have a good bioavailability of the drug. It would also be desirable to provide a composition that can be easily manufactured by a simple process, wherein the risk of product degradation is reduced. Preferably the process avoids the use of process steps that are susceptible to causing polymorphic changes or degradation of the active agent (e.g. melt processing, compaction, and co-precipitation). It would be further desirable to provide a process which can easily be adapted to provide immediate- or modified-release of the active agent. It would be a further desirable if the use of organic solvents and high temperatures are minimized, thus providing environmental and economical advantages. The present invention aims to achieve at least one or more of these objectives.

### SUMMARY OF THE INVENTION

The present invention provides a pharmaceutical dosage form in the form of a tablet comprising:
(a) a compressed inert core
(b) an optional subcoat over the compressed inert core
(c) a drug layer over the compressed core (a) or optional subcoat (b) comprising a drug having a water solubility at 25° C. of about 100 mg/l or less, a coating polymer and optionally a surfactant, and
(d) optionally one or more layers coating the drug layer.

The present invention further provides a process for preparing the pharmaceutical dosage form comprising:
(a) a compressed inert core
(b) an optional subcoat over the compressed inert core
(c) a drug layer over the compressed core (a) or optional subcoat (b) comprising a drug having a water solubility at 25° C. of about 100 mg/l or less, a coating polymer and optionally a surfactant, and
(d) optionally one or more layers coating the drug layer.

### BRIEF DESCRIPTION OF THE FIGURE

FIG. 1 illustrates the dissolution results of tablets containing micronized API (d(0.9) LT 28 or 12.6 μm) with and without a surfactant.

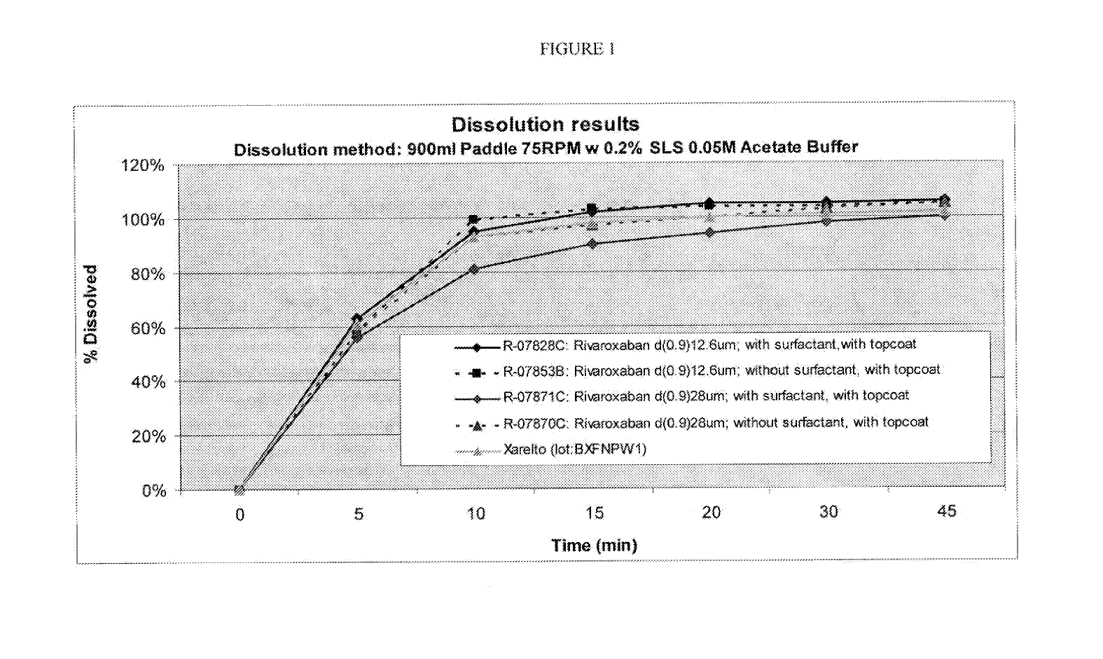

### DETAILED DESCRIPTION OF THE INVENTION

As used herein, unless indicated otherwise, % ranges refer to % by weight. As used herein, unless indicated otherwise, references to the drug include references to pharmaceutically acceptable salts, solvates or hydrates thereof.

As used herein, unless indicated otherwise, references to particle size ranges and particle size distribution ranges [e.g., d(0.9)] and values refer to measurements by laser diffraction, e.g., as obtained using a Malvern Mastersizer 2000.

As used herein, the term “modified release” includes sustained release, controlled release, delayed release, slow release, and extended release. The release rate can be controlled by the use of modified release polymers such those described herein.

The pharmaceutical dosage form of the present invention is a tablet comprising:
(a) a compressed inert core
(b) an optional subcoat over the compressed inert core
(c) a drug layer over the compressed core (a) or optional subcoat (b) comprising a drug having a water solubility at 25° C. of about 100 mg/l or less, a coating polymer and optionally a surfactant, and
(d) optionally one or more layers coating the drug layer.

The dosage form of the present invention may be mono- or multiparticulate. The multiparticulate tablets are typically filled into capsules. Preferably, the dosage form is monoparticulate.

The compressed cores (a) of the dosage form of the present invention preferably comprise a filler, binder, and a lubricant. The cores are inert, i.e., do not comprise a drug. Preferably the compressed core contains less than 5%, preferably less than 2%, more preferably less than 1% disintegrant. Most preferably, the compressed core is free of any disintegrant. Suitable compressed inert cores for use in the pharmaceutical dosage form of the present invention can be prepared by blending a mixture of at least one filler and at least one binder, and further blending in at least one lubricant. The blended mixture can be compressed.

Typically, for the monoparticulate dosage forms of the present invention, the compressed cores can have diameters in the range of 4 to 8 mm, preferably about 4.5 to about 7.5 mm, more preferably about 5.0 to about 7.0 mm, and most preferably about 5.5 to about 6.5 mm (e.g., about 6.0 mm).

The compressed cores provide a stable base for forming the layered compositions of the present invention. Moreover, since the compositions of the present invention comprise layers over a structurally stable core, the final dosage form can be easily adapted to provide modified release dosage forms by applying a modified release coating over and/or within the drug layer. Since the layered cores do not require a final compression step, this avoids the requirement of adding excipients for the purpose of facilitating compression.

In the compositions of the present invention, the compressed inert core is preferably present in the pharmaceutical composition in a range of about 50 to about 85 wt %, preferably about 55 to about 80 wt %, more preferably about 65 to about 75 wt % of the pharmaceutical composition.

Suitable fillers for the core (a) include those selected from the group consisting of microcrystalline cellulose (for example, Avicel PH102 or PH101), lactose in its various forms (e.g., lactose monohydrate, anhydrous or spray dried), sorbitol, dextrose, sucrose, mannitol, dibasic calcium phosphate, starch, and mixtures thereof. Preferred fillers are selected from the group consisting of microcrystalline cellulose, lactose in its various forms (e.g., lactose monohydrate, anhydrous or spray dried), mannitol, dibasic calcium phosphate, starch, and mixtures thereof, particularly, microcrystalline cellulose, mannitol, lactose, and starch and mixtures thereof, and more particularly, microcrystalline cellulose, lactose, and starch, and mixtures thereof. In a particularly preferred embodiment, the filler for the compressed core (a) is selected from the group consisting of microcrystalline cellulose and lactose or mixtures thereof, with a mixture of the two being especially preferred.

Typically, the inert core may contain the filler or mixture of fillers in an amount of about 50 to about 98 wt %, preferably about 60 to about 98 wt %, more preferably about 75 to about 98 wt %, and most preferably about 85 to about 96 wt % or about 90 to about 96 wt %, based on the weight of the inert core.

Suitable binders for the inert core include those selected from the group consisting of: cellulose polymers (such as hydroxypropylmethyl cellulose, hydroxypropyl cellulose, hydroxyethyl cellulose, ethyl cellulose, and methyl cellulose), gelatin, pregelatinized starch, acacia, alginic acid, sodium carboxymethyl cellulose gum arabic, polyvinyl pyrrolidone, polyvinyl alcohol, copolymers of N-vinyl pyrrolidine and vinyl acetate and mixtures thereof. Preferably, the binder is selected from the group consisting of cellulose polymers (such as hydroxypropylmethyl cellulose, hydroxypropylcellulose, methylcellulose and hydroxyethyl cellulose), polyvinylpyrrolidone, polyvinyl alcohol and mixtures thereof, and more preferably the binder is selected from the group consisting of hydroxypropylmethyl cellulose, hydroxypropyl cellulose, hydroxyethyl cellulose, ethyl cellulose, methyl cellulose, polyvinyl pyrrolidone, polyvinyl alcohol, and mixtures thereof. Polyvinyl pyrrolidone is a particularly preferred binder.

The binder may be present in the compressed inert core (a) in a range of about 0.5 to about 20 wt %, preferably about 2 to about 15 wt %, and preferably about 2 to about 10 wt %, and more preferably about 2 to about 8 wt %, based on the weight of the inert core.

Suitable lubricants that can be used in the compressed core (a) include those selected from the group consisting of sodium stearyl fumarate, stearic acid, magnesium stearate, calcium stearate, zinc stearate, talc, glyceryl behenate, hydrogenated vegetable oil, hydrogenated castor oil and mixtures thereof, preferably sodium stearyl fumarate, magnesium stearate, calcium stearate and talc and mixtures thereof. Magnesium stearate is a particularly preferred lubricant for use in the compressed inert cores of the present invention.

The inert cores (a) may contain the lubricant in a concentration range of about 0.05 to about 5 wt %, preferably about 0.1 to about 2 wt %, and preferably about 0.3 to about 0.8 wt % based on the weight of the inert core.

In a particularly preferred embodiment of the present invention, the compressed inert core (a) contains a mixture of microcrystalline cellulose and lactose monohydrate as filler, povidone (preferably PVP K-30) as binder and magnesium stearate as lubricant. Preferably, the compressed inert core (a) contains microcrystalline cellulose and lactose monohydrate in a weight ratio of about 1:1 to about 10:1, preferably about 3:1 to about 8:1, and more preferably about 4:1 to about 6:1.

In any embodiment of the present invention, the compressed inert core can contain a weight ratio of filler (preferably a combination of microcrystalline cellulose and lactose monohydrate) and binder of about 5:1 to about 30:1, preferably about 10:1 to about 25:1, more preferably about 15:1 to about 22:1.

As mentioned above, the present invention provides a means for formulating dosage forms of drugs that have very low water solubility, or drugs that are practically water-insoluble, which avoid at least some of the problems encountered with prior art processes. For example, the present invention enables the production of containing such drugs, wherein the dosage forms have a good blend uniformity of the drug in the drug layer. Moreover, the present invention avoids the need to carry out multiple granulation steps and melting steps, which are not as desirable from the point of view of energy requirements, and drug stability.

Preferably, suitable drugs that can be used in the drug layer (c) of the compositions of any embodiment of the present invention include those drugs having a water solubility at 25° C. of about 80 mg/l or less, preferably about 40 mg/l or less, or about 20 mg/l or less, and more preferably about 10 mg/l or less. The present invention can also be used for drugs that have a water solubility at 25° C. of about 7 mg/l or less.

The present invention is particularly suitable for formulating drugs that are practically insoluble in water, preferably selected from the direct Xa inhibitors. Thus, in a preferred embodiment, the drug is selected from the group consisting of Rivaroxaban, Apixaban, Dabigatran, Ximelagatran, Otamixaban, Edoxaban, and Betrixaban. Rivaroxaban is an especially preferred drug.

In any embodiment of the present invention described herein, the drug is preferably provided in a micronised form, preferably having d(0.9) of less than 100 microns, preferably less than 60 microns, preferably less than 50 microns, preferably less than 40 microns, and more preferably less than 30 microns. Furthermore, it is preferable that the drug has a d(0.9) of from 10 to 30 microns.

The drug loading in the drug layer (c) according to any embodiment of the present invention preferably ranges from about 10 to about 90 wt %, preferably about 20 to about 75 wt %, preferably about 30 to about 60 wt %, and more preferably about 40 to about 50 wt %, based on the weight of the drug layer.

The drug layer (c) comprises at least one coating polymer. The coating polymer is preferably selected from the group consisting of polyvinyl alcohol, cellulose derivatives (such as hydroxypropyl methyl cellulose, hydroxypropyl cellulose, hydroxyethyl cellulose, ethyl cellulose, and methyl cellulose), polymethacrylates (such as Eudragit RS), polyvinylpyrrolidone, polyvinyl alcohol, and mixtures thereof. More preferably, the coating polymer is selected from the group consisting of cellulose derivatives (such as hydroxypropyl methyl cellulose, hydroxypropyl cellulose, hydroxyethyl cellulose, ethyl cellulose, and methyl cellulose), polymethacrylates (such as Eudragit RS), polyvinyl alcohol, and mixtures thereof. Hydroxypropyl methylcellulose and polyvinyl alcohol are particularly preferred. Polyvinyl alcohol is an especially preferred coating polymer for use in the drug layer (c).

Preferably, in any embodiment of the present invention, the drug layer (c) comprises a surfactant. Suitable surfactants include those selected from the group consisting of polyoxyethylene sorbitan fatty acid esters (such as polysorbate 80 or polysorbate 40), polyoxyethylene stearates (such as polyoxyl 40), sodium lauryl sulfate, and sorbitan esters, including sorbitan mono-palmitate, benzalkonium chloride, cetyl alcohol, or mixtures thereof. Preferred surfactants are those selected from the group consisting of polyoxyethylene sorbitan fatty acid esters (such as polysorbate 80 or polysorbate 40), polyoxyethylene stearates (such as polyoxyl 40), and sodium lauryl sulfate and mixtures thereof. Sodium lauryl sulfate is a particularly preferred surfactant for use in the drug layer (c) of the formulations of the present invention.

The surfactant may be present in the drug layer in a concentration of from about 2 to about 20 wt %, preferably about 5 to about 15 wt %, and more preferably about 6 to about 12 wt %, based on the weight of the drug layer.

Preferred drug:surfactant weight ratio ranges in the drug layer are about 1:1 to about 20:1, preferably about 3:1 to about 8:1, and more preferably about 4:1 to about 6:1.

In an alternative embodiment of the invention, the drug layer does not contain a surfactant. In said alternative embodiment, it is preferable that the tablet includes one or more layers coating the drug layer. In said embodiment (excluding a surfactant), all other preferred embodiments of the core and drug layer apply with any amount of surfactant in the drug layer optionally being replaced by the other components of the drug layer.

In any embodiment of the present invention, the drug layer (c) may further comprise a plasticizer. Suitable plasticizers may be selected from the group consisting of triacetin, diethyl phthalate, dibutyl sebacate, tributyl sebacate and polyethylene glycol, and mixtures thereof.

The drug layer (c) may also comprise an anti-adherent or glidant. These are preferably selected from the group consisting of talc, fumed silica, and magnesium stearate. Talc is a particularly preferred anti-adherent/glidant.

The drug layer can also comprise further excipients, such as an opacifying agent (preferably titanium dioxide), and optionally a colourant, preferably an iron oxide based colourant, or a mixture thereof.

Conveniently, the coating polymer component of the drug layer may be provided by the use of a commercially available fully-formulated pharmaceutical film coating systems such as Opadry® coating systems (Colourcon). For example, the drug layer can comprise the drug, and preferably a surfactant, in combination with a commercially available film coating system e.g. containing hypromellose and/or Polyvinyl alcohol (e.g. Opadry®, Opadry® HP, Opadry® II or Opadry AMB®).

It has surprisingly been found that dosage forms of the present invention can achieve good release rate and bioavailability of the poorly water soluble drug without the need for disintegrants. In this regard, it is preferred that the drug layer (c) contains less than 5%, preferably less than 2%, more preferably less than 1% disintegrant. In the preferred embodiments of any aspect of the invention, the drug layer (c) is free of any disintegrant.

Moreover, the applicant has found that the layered compositions of the present invention are able to achieve good release rate and bioavailability of the drug without the need to use pseudoemulsifiers such as those required in the formulations of WO 2010/003641. This is particularly advantageous since the pseudoemulsifiers required in the prior art formulations are natural products (in particular natural gums) which inherently contain an equilibrium amount of water, which may give rise to potential drug-excipient interactions. In turn, this may lead to storage stability issues. This is further exacerbated by the fact that natural products such as gums vary in terms of chemical content, purity and hydration state. For a low dose drug, such as the factor Xa inhibitors and in particular rivaroxaban, storage stability is of particular importance.

The layered cores of the present invention can be readily adapted to produce a dosage form having a modified release of the drug. In one embodiment, the modified forms of the present invention can be provided by the inclusion of a modified release polymer in the drug layer (c). Such modified release polymers include those selected from the group consisting of ethyl cellulose, methacrylate copolymers (e.g., Eudragit L30 D55—an anionic polymethacrylate), hydroxypropylmethyl cellulose phthalate, hydroxypropylmethyl cellulose acetate succinate and polyvinylacetate phthalate. Ethyl cellulose is a preferred modified release polymer. Ethyl cellulose having various viscosity grades can be used in order to provide specific release characteristics. For example, ethyl cellulose having viscosity grades of 7, 10, 50, and 100 cPs can be used. Preferably ethyl cellulose having a viscosity grade of 7 cPs is used to provide an extended release of the drug.

In any embodiment of the present invention, the drug layer (c) can be separated from the compressed core (a) by the inclusion of an intermediate subcoat (b) disposed between the core (a) and drug layer (c).

The subcoat preferably comprises a coating polymer and optionally one or more excipients selected from the group consisting of a plasticizer, an anti-adherent or glidant, an opacifying agent, and a colourant.

The subcoat may further comprise a plasticizer, preferably selected from the group consisting of triacetin, diethyl phthalate, dibutyl sebacate, tributyl sebacate and polyethylene glycol, and mixtures thereof.

The subcoat may additional comprise an anti-adherent or glidant, preferably selected from the group consisting of talc, fumed silica, magnesium stearate.

A suitable subcoat may be provided by a commercially available fully-formulated pharmaceutical film coating systems such as Opadry® coating systems (Colourcon) as described above for the drug layer (b). A particularly preferred fully-formulated film coating system for the subcoat can comprise a coating polymer as described above, a plasticizer as described above, and one or more pigments. For example, a suitable subcoat can contain hypromellose and/or polyvinyl alcohol, polyethylene glycol, and one or more pigments such as titanium dioxide and iron oxide based pigments (e.g. Opadry® II film coating system).

In the dosage forms of the present invention the drug layer (c) may be further coated with a coating layer (d). For example, layer (d) may comprise a protective top coat which is disposed over the drug layer (c). In this embodiment, the protective top coat layer (d) preferably comprises a coating polymer and optionally one or more excipients selected from the group consisting of a plasticizer, an anti-adherent or glidant, and one or more pigments/opacifying agents.

Suitable plasticizers for the protective top coat are preferably selected from the group consisting of triacetin, diethyl phthalate, dibutyl sebacate, tributyl sebacate and polyethylene glycol, and mixtures thereof. Polyethylene glycol is a preferred plasticizer.

Suitable anti-adherent or glidants for the protective top coat are preferably selected from the group consisting of talc, fumed silica, magnesium stearate, and more preferably talc.

As an alternative to the inclusion of a modified release polymer in the drug layer (c), compositions providing a modified release of the drug can be formulated by the provision of a modified release layer as layer (d′). The modified release layer (d′) is disposed over the drug layer (c) or over the protective top coat (d).

The modified release layer (d′) comprises a modified release polymer and optionally an excipient selected from the group consisting of a plasticizer and a pore former, or a mixture thereof. Preferably the modified release layer (d′) comprises a modified release polymer, a plasticizer, and a pore former.

More preferably, the modified release polymer is as described above for the drug layer (c), i.e. is selected from the group consisting of ethyl cellulose, methacrylate copolymers (e.g., Eudragit L30 D55—an anionic polymethacrylate), hydroxypropylmethyl cellulose phthalate, hydroxypropylmethyl cellulose acetate succinate and polyvinylacetate phthalate. A dosage form according to embodiment 59, wherein the modified release polymer is ethyl cellulose. Ethyl cellulose having various viscosity grades can be used in order to provide specific release characteristics for the modified release layer. For example, ethyl cellulose having viscosity grades of 7, 10, 50, and 100 cPs can be used. Preferably ethyl cellulose having a viscosity grade of 7 cPs is used to provide an extended release of the drug.

Suitable plasticizers for layer (d′) comprise those selected from the group consisting of triethyl citrate, tributyl citrate, glycerin, dibutyl sebacate, triacetin and diethylphthalate, or mixtures thereof, with triethyl citrate, tributyl citrate, dibutyl sebacate, and diethyl phthalate, or mixtures thereof being preferred. Dibutyl sebacate is particularly preferred.

Suitable pore formers for use in the modified release layer (d′) include hydroxypropyl methyl cellulose (preferably HPMC 6 cPs) and polyethylene glycol (preferably PEG 400), or a mixture thereof.

The modified release layer (d′) may be further provided with a protective top coat, which can be comprised of the components as described for the protective top coat over drug layer (c).

In preferred embodiments of any aspect of the present invention, the compressed core (a) contains less than 5%, preferably less than 2%, more preferably less than 1% disintegrant, relative to the weight of the dosage form, and most preferably wherein the compressed core is free of any disintegrant. In preferred embodiments of any aspect of the present invention, the drug layer (c) contains less than 5%, preferably less than 2%, more preferably less than 1% disintegrant, relative to the weight of the dosage form, and most preferably wherein the drug layer is free of any disintegrant.

In preferred embodiments of any aspect of the present invention, the dosage forms as described above contain less than 5%, preferably less than 2%, more preferably less than 1%, particularly less than 0.2% disintegrant, relative to the weight of the dosage form. In a particularly preferred embodiment, the dosage form is free of any disintegrant.

The dosage forms of any embodiment of the present invention can be prepared by a process comprising:
(a) obtaining a compressed inert core,
(b) optionally applying a subcoat over the compressed inert core,
(c) applying the drug layer over the compressed core (a) or optional subcoat (b), and optionally
(d) applying one or more layers over the drug layer.

The compressed core can be obtained by a process as described above. For example the compressed core can be prepared by:

(i) blending a mixture comprising a filler, binder and lubricant, and

(ii) direct compression of the mixture to obtain the compressed core.

The blending step (i) can be carried out in stages, i.e. first blending the filler and binder to form a mixture, and subsequently blending a lubricant into the mixture. The filler, binder, and lubricant components are as defined above.

If the optional sub-coat (b) is required as an intermediate layer between the inert core (a) and drug coat (c), this can be applied by preparing a solution, dispersion or suspension of the coating excipients as described in any of the above embodiments for the sub-coat (c) in a solvent. The solvent can be any pharmaceutically acceptable solvent such as water, acetone, ethanol, or isopropanol. Preferably the solvent is water. Conveniently the coating medium is a 1-40% w/w, preferably 5-20% w/w, more preferably 5-15 wt %, and typically about 10% w/w solution, dispersion, or suspension. Preferably the sub-coating medium comprises an aqueous dispersion of the coating excipients as described above. The coating is typically carried out using any suitable means for applying a coating, such as spraying, e.g., using a pan coater.

The drug layer (c) can be applied by preparing a dispersion, suspension or solution comprising the drug (preferably wherein the drug is in micronized form (preferably having the preferred particle size distributions as described above), coating polymer (preferably wherein the coating polymer is as defined in any of the above embodiments), and optionally a surfactant (preferably wherein the surfactant is as defined in any of the above embodiments) in a pharmaceutically acceptable solvent as described above for the sub-coat (b). Preferably the drug coating medium is in the form of an aqueous dispersion comprising the micronized drug, coating polymer and optionally a surfactant. As indicated above, the drug coating medium may also comprise excipients to provide a modified release. These may be added to the drug coating medium. Conveniently the drug coating medium is a 1-40% w/w, preferably 5-20% w/w, more preferably 5-15 wt %, and typically about 10% w/w solution, dispersion, or suspension. The dispersion, suspension or solution can be sprayed over the inert core (a), or the subcoat (b) if present, by using any suitable means for applying a coating, such as spraying, e.g. using a pan coater.

As discussed above, the drug layer (c) may be provided with a protective top coat (d). The protective top coat (d) can be applied by preparing a solution, dispersion, or suspension of the coating excipients as described in any of the above embodiments for the sub-coat (d) in a solvent. The solvent can be any pharmaceutically acceptable solvent such as water, acetone, ethanol, or isopropanol. Preferably the solvent is water. Conveniently the coating medium is a 1-40% w/w, preferably 5-20% w/w, more preferably 5-15 wt %, and typically about 10% w/w solution, dispersion, or suspension. Preferably the coating medium comprises an aqueous dispersion of the coating excipients as described above. The coating is typically carried out using any suitable means for applying a coating, such as spraying, e.g. using a pan coater.

As an alternative to using a modified release polymer in drug layer (c), a modified release layer (d′) may be provided over the drug layer (c), or the protective top coat (d). The modified release layer (d′) can be applied by preparing a solution, dispersion or suspension of the excipients as described in any of the above embodiments for the modified release layer (d′) in a solvent. The solvent can be any pharmaceutically acceptable solvent such as water, acetone, ethanol, or isopropanol. Preferably the solvent is ethanol, acetone or water or a mixture thereof (more preferably a mixture of these). Conveniently the coating medium is a 1-40% w/w, preferably 2-20% w/w, more preferably 5-10 wt %, and typically about 7% w/w solution, dispersion, or suspension. Preferably the coating medium comprises solution of the excipients as described above for modified release layer (d′). The coating is typically carried out using any suitable means for applying a coating, such as spraying, e.g. using a pan coater.

It can be seen that the pharmaceutical dosage forms of the present invention can be manufactured by a simple and economical processes, wherein the use of organic solvents and high temperatures are minimized. Moreover, the layered structure of the dosage form enables easy adaptation of the formulation to suit particular characteristics of the active agent.

For the avoidance of doubt, additional embodiments of the present invention include those where each use of the term “comprising” is replaced with “consisting of” or “consisting essentially of” with such terms having their generally accepted meanings.

The present invention will now be described with reference to the following examples, which serve to illustrate the various embodiments of the present invention and which are not intended to be limiting. The skilled person will appreciate that modifications are within the spirit and scope of the invention.

### EXAMPLE

### Example 1

### Inert Placebo Core Coated with Rivaroxaban Drug Layer

Microcrystalline cellulose, Lactose monohydrate, and Povidone (PVP K-30) are combined into a blend using a diffusion blender for 5 min. The mixture obtained is then blended with magnesium stearate for an additional 3 min. The final mixture is compressed into 6.0 mm tablets by a rotary tablet press.

Drug coating dispersion is made by dispersing micronized API (d(0.9) LT 30 μm), sodium lauryl sulfate and Opadry in purified water in order to achieve 10% (w/w) solids dispersion. The dispersion is then sprayed over inert placebo cores using a pan coater. The dispersion is mixed during the coating process.

Formulation of tablets of Example 1 by weight

| Component | Function | mg/tab | Layer |
|---|---|---|---|
| MICROCRYSTALLINE | Filler | 47.7 | INERT |
| CELLULOSE | | | PLACEBO |
| LACTOSE MONOHYDRATE | Filler | 9.0 | CORE |
| SPRAY DRIED | | | |
| POVIDONE (PVP K-30) | Binder | 3.0 | |
| MAGNESIUM STEARATE | Lubricant | 0.3 | |
| TOTAL INERT PLACEBO CORE WEIGHT | | 60.0 | |
| RIVAROXABAN MICRONIZED | Active | 10.0 | DRUG |
| (d(0.9) LT 30 um) | | | LAYER |
| SODIUM LAURYL SULFATE | Surfactant | 2.0 | |
| OPADRY ® (hypromellose | Coating | 10.0 | |
| and/or polyvinyl alcohol based | polymer | | |
| ready made formulation) | | | |
| PURIFIED WATER | Process | Removed during | |
| | Solvent | process | |

| | |
|---|---|
| TOTAL DRUG COATED TABLET WEIGHT | 82.0 |

Note:
*OPADRY ® & OPADRY ® II are fully-formulated coating systems manufactured by Colorcon.

### Example 2

### Inert Placebo Core Coated with Rivaroxaban Drug Layer and a Protective Layer

Drug coated tablets are prepared as described in Example 1. A top (i.e., protective) coating dispersion is made by dispersing Opadry in purified water in order to achieve 10% (w/w) solids dispersion. The dispersion is then sprayed over the drug coated placebo cores using a pan coater. The dispersion is mixed during the coating process.

Formulation of tablets of Example 2 by weight

| Component | Function | mg/tab | Layer |
|---|---|---|---|
| MICROCRYSTALLINE | Filler | 47.7 | INERT |
| CELLULOSE | | | PLACEBO |
| LACTOSE MONOHYDRATE | Filler | 9.0 | CORE |
| SPRAY DRIED | | | |
| POVIDONE (PVP K-30) | Binder | 3.0 | |
| MAGNESIUM STEARATE | Lubricant | 0.3 | |
| TOTAL INERT PLACEBO CORE WEIGHT | | 60.0 | |
| RIVAROXABAN MICRONIZED | Active | 10.0 | DRUG |
| (d(0.9) LT 30 um) | | | LAYER |
| SODIUM LAURYL SULFATE | Surfactant | 2.0 | |
| OPADRY ®(hypromellose and/or | Coating | 10.0 | |
| polyvinyl alcohol based ready | polymer | | |
| made formulation) | | | |
| PURIFIED WATER | Process | Removed | |
| | Solvent | during process | |
| TOTAL DRUG COATED TABLET WEIGHT | | 82.0 | |
| OPADRY ® (hypromellose | Coating | 3.0 | TOP |
| and/or polyvinyl alcohol based | polymer | | COAT |
| ready made formulation) | | | |
| PURIFIED WATER | Process | Removed | |
| | Solvent | during process | |

| | |
|---|---|
| TOTAL DRUG COATED TABLET WEIGHT | 85.0 |

### Example 3

### Inert Placebo Core Coated with Rivaroxaban Drug Layer

Microcrystalline cellulose, Lactose monohydrate, and Povidone (PVP K-30) were combined into a blend using a diffusion blender for 5 min. The mixture obtained was then blended with magnesium stearate for an additional 3 min. The final mixture was compressed into 6.0 mm tablets by a rotary tablet press.

Drug coating dispersion was made by dispersing micronized API (d(0.9) LT 30 μm), sodium lauryl sulfate and Opadry in purified water in order to achieve 10% (w/w) solids dispersion. The dispersion was then sprayed over inert placebo cores using a pan coater. The dispersion was mixed during the coating process.

Formulation of tablets of Example 3 by weight

| Component | Function | mg/tab | Layer |
|---|---|---|---|
| MICROCRYSTALLINE | Filler | 47.7 | INERT |
| CELLULOSE | | | PLACEBO |
| LACTOSE MONOHYDRATE | Filler | 9.0 | CORE |
| SPRAY DRIED | | | |
| POVIDONE (PVP K-30) | Binder | 3.0 | |
| MAGNESIUM STEARATE | Lubricant | 0.3 | |
| TOTAL INERT PLACEBO CORE WEIGHT | | 60.0 | |
| RIVAROXABAN MICRONIZED | Active | 10.0 | DRUG |
| (d(0.9) LT 30 um) | | | LAYER |
| SODIUM LAURYL SULFATE | Surfactant | 2.0 | |
| OPADRY ® (hypromellose | Coating | 10.0 | |
| and/or polyvinyl alcohol based | polymer | | |
| ready made formulation) | | | |
| PURIFIED WATER | Process | Removed during | |
| | Solvent | process | |

| | |
|---|---|
| TOTAL DRUG COATED TABLET WEIGHT | 82.0 |

Note:
*OPADRY ® & OPADRY ® II are fully-formulated coating systems manufactured by Colorcon.

Following this method tablets containing micronized API (d_{(0.9)} LT 12.6 μm) were also prepared.

### Example 4

### Inert Placebo Core Coated with Rivaroxaban Drug Layer and a Protective Layer

Drug coated tablets were prepared as described in Example 3. A top (i.e., protective) coating dispersion was made by dispersing Opadry in purified water in order to achieve 10% (w/w) solids dispersion. The dispersion was then sprayed over the drug coated placebo cores using a pan coater. The dispersion was mixed during the coating process.

Formulation of tablets of Example 4 by weight

| Component | Function | mg/tab | Layer |
|---|---|---|---|
| MICROCRYSTALLINE | Filler | 47.7 | INERT |
| CELLULOSE | | | PLACEBO |
| LACTOSE MONOHYDRATE | Filler | 9.0 | CORE |
| SPRAY DRIED | | | |
| POVIDONE (PVP K-30) | Binder | 3.0 | |
| MAGNESIUM STEARATE | Lubricant | 0.3 | |
| TOTAL INERT PLACEBO CORE WEIGHT | | 60.0 | |
| RIVAROXABAN MICRONIZED | Active | 10.0 | DRUG |
| (d(0.9) LT 30 um) | | | LAYER |
| SODIUM LAURYL SULFATE | Surfactant | 2.0 | |
| OPADRY ®(hypromellose and/or | Coating | 10.0 | |
| polyvinyl alcohol based ready | polymer | | |
| made formulation) | | | |
| PURIFIED WATER | Process | Removed | |
| | Solvent | during process | |
| TOTAL DRUG COATED TABLET WEIGHT | | 82.0 | |
| OPADRY ® (hypromellose | Coating | 3.0 | TOP |
| and/or polyvinyl alcohol based | polymer | | COAT |
| ready made formulation) | | | |
| PURIFIED WATER | Process | Removed | |
| | Solvent | during process | |

| | |
|---|---|
| TOTAL DRUG COATED TABLET WEIGHT | 85.0 |

Following this method, tablets containing micronized API (d(0.9) LT 28 or 12.6 μm) may be prepared with and without a surfactant. Batches were prepared having the composition given above with the variations given below;

| | | |
|---|---|---|
| R7828C | d_{(0.9)} LT 12.6 μm | With surfactant |
| | | (2.0 mg/tablet SLS) |
| R7853B | d_{(0.9)} LT 12.6 μm | no surfactant |
| R7871C | d_{(0.9)} LT 28 μm | With surfactant |
| | | (2.0 mg/tablet SLS) |
| R7870C | d_{(0.9)} LT 28 μm | no surfactant |

### Example 5

### Inert Placebo Core Coated with Inner Seal Coat, Rivaroxaban Drug Layer and a Protective Layer

Inert placebo cores are prepared as described in Example 1. Inner seal coat (subcoat) coating dispersion is made by dispersing Opadry in purified water in order to achieve 10% (w/w) solids dispersion. The dispersion is then sprayed over the inert placebo cores using a pan coater. The seal coat dispersion is mixed during the coating process.

The drug coating dispersion is made by dispersing micronized API (d(0.9) LT 30 μm), sodium lauryl sulfate and Opadry in purified water in order to achieve 10% (w/w) solids dispersion. The dispersion is then sprayed over inner seal coat coated tablets using a pan coater. The drug coating dispersion is mixed during the coating process.

A top coat (i.e., protective) layer is prepared is made by dispersing Opadry in purified water in order to achieve 10% (w/w) solids dispersion. The dispersion is then sprayed over the drug coated placebo cores using a pan coater. The dispersion is mixed during the coating process.

Formulation of tablets of Example 5 by weight

| Component | Function | mg/tab | Layer |
|---|---|---|---|
| MICROCRYSTALLINE | Filler | 47.7 | INERT |
| CELLULOSE | | | PLACEBO |
| LACTOSE MONOHYDRATE | Filler | 9.0 | CORE |
| SPRAY DRIED | | | |
| POVIDONE (PVP K-30) | Binder | 3.0 | |
| MAGNESIUM STEARATE | Lubricant | 0.3 | |
| TOTAL INERT PLACEBO CORE WEIGHT | | 60.0 | |
| OPADRY ® (hypromellose and/or | Coating | 2.5 | INNER |
| polyvinyl alcohol based ready made | polymer | | SEAL |
| formulation) | | | COAT |
| PURIFIED WATER | Process Solvent | Removed | (SUB- |
| | | during process | COAT) |
| TOTAL INNER SEAL COATED TABLET WEIGHT | | 62.5 | |
| RIVAROXABAN MICRONIZED | Active | 10.0 | DRUG |
| (d(0.9) LT 30 um) | | | LAYER |
| SODIUM LAURYL SULFATE | Surfactant | 2.0 | |
| OPADRY ® (hypromellose and/or | Coating | 10.0 | |
| polyvinyl alcohol based ready made | polymer | | |
| formulation) | | | |
| PURIFIED WATER | Process Solvent | Removed | |
| | | during process | |
| TOTAL DRUG COATED TABLET WEIGHT | | 84.5 | |
| OPADRY ®(hypromellose and/or | Coating | 3.0 | TOP |
| polyvinyl alcohol based ready made | polymer | | COAT |
| formulation) | | | |
| PURIFIED WATER | Process Solvent | Removed | |
| | | during process | |
| TOTAL TOP COATED TABLET WEIGHT | | 87.5 | |

### Example 6

### Inert Placebo Core Coated with Inner Seal Coat, Rivaroxaban Drug Layer, and an Extended Release Coat

Inert placebo cores are prepared as described in Example 1. Inner seal coat (subcoat) coating dispersion is made by dispersing Opadry in purified water in order to achieve 10% (w/w) solids dispersion. The dispersion is then sprayed over the inert placebo cores using a pan coater. The seal coat dispersion is mixed during the coating process.

The drug coating dispersion is made by dispersing micronized API (d(0.9) LT 30 μm), sodium lauryl sulfate and Opadry in purified water in order to achieve 10% (w/w) solids dispersion. The dispersion is then sprayed over inner seal coat coated tablets using a pan coater. The drug coating dispersion is mixed during the coating process.

An extended release coating solution is made by mixing ethanol, and Acetone. Ethyl cellulose (ETHYLCELLULOSE PREMIUM 7 CPS) is then added to the aforementioned mixture and is mixed until fully dissolved. Purified water is then added to the solution while mixing at high speed, and then DIBUTYL SEBACATE and HYPROMELLOSE 6 cPs are added. The solvent ratio is 2:1:1 (alcohol:acetone:purified water), and the percentage of solids in the solution is 7% (w/w). The dispersion is then sprayed over the drug coated placebo cores using a pan coater. The extended release coating dispersion is mixed during the coating process. The extended release coat may be followed by a colored protective layer as described in Examples 1, 2 and 3.

Formulation of tablets of Example 6 by weight

| Component | Function | mg/tab | Layer |
|---|---|---|---|
| MICROCRYSTALLINE | Filler | 47.7 | INERT |
| CELLULOSE | | | PLACEBO |
| LACTOSE MONOHYDRATE | Filler | 9.0 | CORE |
| SPRAY DRIED | | | |
| POVIDONE (PVP K-30) | Binder | 3.0 | |
| MAGNESIUM STEARATE | Lubricant | 0.3 | |
| TOTAL INERT PLACEBO CORE WEIGHT | | 60.0 | |
| OPADRY ®(hypromellose | Coating | 2.5 | INNER SEAL |
| and/or polyvinyl alcohol | polymer | | COAT |
| based ready made | | | (SUB-COAT) |
| formulation) | | | |
| PURIFIED WATER | Process | Removed during | |
| | Solvent | process | |
| TOTAL INNER SEAL COAT WEIGHT | | 62.5 | |
| RIVAROXABAN MICRONIZED | Active | 10.0 | DRUG |
| (d(0.9) LT 30 um) | | | LAYER |
| SODIUM LAURYL | Surfactant | 2.0 | |
| SULFATE | | | |
| OPADRY ®(hypromellose | Coating | 10.0 | |
| and/or polyvinyl alcohol | polymer | | |
| based ready made | | | |
| formulation) | | | |
| PURIFIED WATER | Process | Removed during | |
| | Solvent | process | |
| TOTAL DRUG COATED TABLET WEIGHT | | 84.5 | |
| OPADRY ®(hypromellose | Coating | 3.0 | TOP |
| and/or polyvinyl alcohol | polymer | | COAT |
| based ready made | | | |
| formulation) | | | |
| PURIFIED WATER | Process | Removed during | |
| | Solvent | process | |
| TOTAL DRUG COATED TABLET WEIGHT | | 87.5 | |
| ETHYLCELLULOSE | Extended | 7.4 | EXTENDED |
| PREMIUM 7 CPS | release | | RELEASE |
| | polymer | | COAT |
| DIBUTYL SEBACATE | plasticizer | 1.1 | |
| HYPROMELLOSE 6 cPs | pore-former | 1.0 | |
| ACETONE | Process | Removed during | |
| | Solvent | process | |
| Ethanol | Process | Removed during | |
| | Solvent | process | |
| PURIFIED WATER | Process | Removed during | |
| | Solvent | process | |

| | |
|---|---|
| TOTAL EXTENDED RELEASE COATED TABLET WEIGHT | 97.0 |

What is claimed is:

1. A pharmaceutical dosage form in the form of a tablet comprising:
(a) a compressed inert core,
(b) an optional subcoat over the compressed inert core,
(c) a drug layer over the compressed core (a) or optional subcoat (b) comprising a drug having a water solubility at 25° C. of about 100 mg/l or less, a coating polymer and optionally a surfactant, and
(d) optionally one or more layers coating the drug layer.

2. A dosage form according to claim 1, wherein the compressed inert core (a) comprises a filler, a binder, and a lubricant.

3. A dosage form according to claim 1 wherein the compressed inert core is present in the pharmaceutical composition in a range of about 50 to about 85 wt %, preferably about 55 to about 80 wt %, more preferably about 65 to about 75 wt % of the pharmaceutical composition.

4. A dosage form according to claim 2 wherein the filler is selected from the group consisting of microcrystalline cellulose (for example, Avicel PH102 having or PH101), lactose in its various forms (e.g. lactose monohydrate, anhydrous or spray dried), sorbitol, dextrose, sucrose, mannitol, dibasic calcium phosphate, starch, and mixtures thereof.

5. A dosage form according to claim 2 wherein the binder is selected from the group consisting of: cellulose polymers (such as hydroxypropylmethyl cellulose, hydroxypropyl cellulose, hydroxyethyl cellulose, ethyl cellulose, and methyl cellulose), gelatin, pregelatinized starch, acacia, alginic acid, sodium carboxymethyl cellulose gum arabic, polyvinyl pyrrolidone, polyvinyl alcohol, and copolymers of N-vinyl pyrrolidine and vinyl acetate or mixtures thereof.

6. A dosage form according to claim 2 wherein the lubricant in the compressed core (a) is selected from the group consisting of sodium stearyl fumarate, stearic acid, magnesium stearate, calcium stearate, zinc stearate, talc, glyceryl behenate, hydrogenated vegetable oil, hydrogenated castor oil and mixtures thereof.

7. A dosage form according to claim 2 wherein the compressed inert core contains a mixture of microcrystalline cellulose and lactose monohydrate as filler, povidone (preferably PVP K-30) as binder and magnesium stearate as lubricant.

8. A dosage form according to claim 1 wherein the drug layer (c) comprises a drug having a water solubility at 25° C. of about 80 mg/l or less.

9. A dosage form according to claim 1 wherein the drug layer comprises a drug having a water solubility of at 25° C. of about 40 mg/l or less.

10. A dosage form according to claim 1 wherein the drug layer comprises a drug having a water solubility of at 25° C. of about 20 mg/l or less, and more preferably about 10 mg/l or less.

11. A dosage form according to claim 1 wherein the drug is an anticoagulant selected from the factor Xa inhibitors (such as Rivaroxaban, Apixaban, Ximelagatran, Otamixaban, Edoxaban, Betrixaban).

12. A dosage form according to claim 1 wherein the drug is Rivaroxaban.

13. A dosage form according to claim 1 wherein the drug is having d(0.9) of less than 100 microns.

14. A dosage form according to claim 13 wherein the drug is having d(0.9) of less than 50 microns.

15. A dosage form according to claim 13 wherein the drug is having d(0.9) of less than 30 microns.

16. A dosage form according to claim 1 wherein the drug is present in the drug layer in an amount of from about 10 to about 90 wt % based on the weight of the drug layer.

17. A dosage form according to claim 1 wherein the drug is present in the drug layer in an amount of from about 30 to about 60 wt % based on the weight of the drug layer.

18. A dosage form according to claim 1 wherein the drug is present in the drug layer in an amount of from about 40 to about 50 wt % based on the weight of the drug layer.

19. A dosage form according to claim 1 wherein the drug layer contains a surfactant.

20. A dosage form according to claim 19 wherein the surfactant in the drug layer (c) is selected from the group consisting of polyoxyethylene sorbitan fatty acid esters (such as polysorbate 80 or polysorbate 40), polyoxyethylene stearates (such as polyoxyl 40), sodium lauryl sulfate, sorbitan esters including sorbitan mono-palmitate, benzalkonium chloride, cetyl alcohol, or mixtures thereof.

21. A dosage form according to claim 1 wherein the coating polymer in drug layer (c) is selected from the group consisting of polyvinyl alcohol, cellulose derivatives (such as hydroxypropyl methyl cellulose, hydroxypropyl cellulose, hydroxyethyl cellulose, ethyl cellulose, methyl cellulose), polymethacrylates (such as Eudragit RS), polyvinylpyrrolidone, polyvinyl alcohol and mixtures thereof.

22. A dosage form according to claim 1 wherein the drug layer (c) further comprises a plasticiser selected from the group consisting of triacetin, diethyl phthalate, dibutyl sebacate, tributyl sebacate and polyethylene glycol, and mixtures thereof.

23. A dosage form according to claim 1 wherein the drug layer (c) further comprises an anti-adherent or glidant selected from the group consisting of talc, fumed silica, and magnesium stearate.

24. A dosage form according to claim 1 wherein the drug layer further comprises a modified release polymer.

25. A dosage form according to claim 24 wherein the modified release polymer is selected from the group consisting of ethyl cellulose, methacrylate copolymers (e.g. Eudragit L30 D55—an anionic polymethacrylate), hydroxypropylmethyl cellulose phthalate, hydroxypropylmethyl cellulose acetate succinate and polyvinylacetate phthalate.

26. A dosage form according to claim 1 comprising a subcoat (b) disposed between the compressed inert core (a) and the drug layer (c).

27. A dosage form according to claim 26 wherein the subcoat comprises a coating polymer and optionally one or more excipients selected from the group consisting of: a plasticiser, an anti-adherent or glidant, an opacifying agent, and a colourant.

28. A dosage form according to claim 26 wherein the subcoat comprises a plasticiser selected from the group consisting of triacetin, diethyl phthalate, dibutyl sebacate, tributyl sebacate and polyethylene glycol, and mixtures thereof.

29. A dosage form according to claim 26 wherein the subcoat comprises an anti-adherent or glidant selected from the group consisting of talc, fumed silica and magnesium stearate.

30. A dosage form according to claim 1 wherein the layer (d) is a protective top coat disposed over the drug layer (c).

31. A dosage form according to claim 30 wherein the protective top coat comprises a coating polymer and optionally one or more excipients selected from the group consisting of: a plasticiser, an anti-adherent or glidant, an opacifying agent, and a colourant.

32. A dosage form according to claim 1 wherein the layer (d) comprises a modified release layer (d′).

33. A dosage form according to claim 32 wherein the modified release layer (d′) is disposed over the drug layer (c) or over the protective top coat (d).

34. A dosage form according to claim 32 wherein the modified release layer is further coated with a protective top coat.

35. A dosage form according to claim 1 wherein the compressed core (a) contains less than 5% disintegrant, relative to the weight of the dosage form.

36. A dosage form according to claim 1 wherein the compressed core is free of any disintegrant.

37. A dosage form according to claim 1 wherein the drug layer (c) contains less than 5% disintegrant, relative to the weight of the dosage form.

38. A dosage form according to claim 1 wherein the drug layer is free of any disintegrant.

39. A dosage form according to claim 1 which contains less than 5% disintegrant, relative to the weight of the dosage form.

40. A dosage form according to claim 1 wherein the dosage form is free of any disintegrant.

41. A dosage form according to claim 1 wherein the drug layer does not contain a surfactant.

42. A non-compressed dosage form according to claim 1.

43. A process for preparing a dosage form as defined in claim 1 comprising:
(a) obtaining a compressed inert core,
(b) optionally applying a subcoat over the compressed inert core,
(c) applying the drug layer over the compressed core (a) or optional subcoat (b), and optionally
(d) applying one or more layers over the drug layer.

44. A process according to claim 43 wherein step (a) comprises:
(i) blending a mixture comprising a filler, binder and lubricant, and
(ii) direct compression of the mixture to obtain the compressed core.

45. A process according to claim 43 wherein the compressed inert core contains less than 5% disintegrant, relative to the weight of the dosage form.

46. A process according to claim 43 wherein the compressed inert core is free of any disintegrant.
